(19) United States
(12) Patent Application Publication
(10) Pub. No.: US 2007/0224281 A1
(43) Pub. Date: Sep. 27, 2007

(54) Sustained-Release Preparations Containing Topiramate and the Producing Method Thereof

(75) Inventors: Jin Woo Park, Seoul (KR); Young Hee Shin, Gyeonggi-do (KR); Kwang Hyun Shin, Gyeonggi-do (KR); Jung Ju Kim, Gyeonggi-do (KR)

Correspondence Address: HESLIN ROTHENBERG FARLEY & MESITI PC, 5 COLUMBIA CIRCLE, ALBANY, NY, 12203, US

(73) Assignee: AMOREPACIFIC CORPORATION, Seoul (KR)

(21) Appl. No.: 11/572,326

(22) PCT Filed: Jul. 21, 2005
(86) PCT No.: PCT/KR05/02361

§ 371 Date: Jan. 19, 2007

(30) Foreign Application Priority Data
Jul. 22, 2004 (KR) 10-2004-0057239

(51) Int. Cl. A61K 9/16

(52) U.S. Cl. 424/495

(57) ABSTRACT

Disclosed herein is a sustained-release topiramate preparation and a method for producing the topiramate preparation. The sustained-release topiramate preparation is produced using double granules obtained by granulating topiramate or a pharmaceutically acceptable salt thereof using a solid dispersant by a solid dispersion method (first granulation), and further by granulating the granules using a release sustaining material by dry or wet granulation (second granulation).

### TECHNICAL FIELD

The present invention relates to sustained-release preparations containing topiramate and a method for producing the preparations.

### BACKGROUND ART

Topiramate is an anticonvulsant drug that has a water solubility as low as 9.8 mg/mL. Since commercially available topiramate preparations are rapidly disintegrated after oral administration, patients experience no serious side effects in the dissolution and absorption of the drug. However, the topiramate preparations cause adverse side effects due to rapid absorption and increased blood level of the drug, and have inconvenience for patients due to oral administration twice daily. In view of these disadvantages, there exists a need for a sustained-release preparation of topiramate.

Immediate-release preparations achieve their pharmacological activity immediately after administration, whereas sustained-release preparations achieve their pharmacological activity over a long period of time. Particularly, in the case of antipsychotic drugs for long-term treatment, more than half of psychopathic patients feel inconvenience due to frequent administration of the drugs, which becomes the major cause of treatment failure. Since it is inevitable that topiramate preparations on the market are accompanied with repeated administration, the inconvenience of patients grows heavier. Sustained-release topiramate preparations can provide convenience of administration by reducing the frequency of daily administration.

In general, the blood drug concentrations are controlled by delayed drug absorption through control of the release of the drugs from drug preparations when there are no particular limitations to the dissolution and absorption of the drugs in the gastrointestinal tract. Specifically, in the case of highly water-soluble drugs, pellets containing the drugs are coated with a release delay layer or mixed with a hydrophobic substance to produce matrix tablets in order to control the diffusion of the drugs dissolved in the preparations, thus achieving sustained-release properties for the drugs. Typical sustained-release preparations include coated pellets, tablets and capsules. The release profiles of drugs through such preparations depend on particular characteristics of the preparations, such as selective breakdown of coating layers and swelling of inner matrices.

In the case where simple matrix tablets are applied to highly water-soluble drugs, there are problems that hydrophobic release delay additives are required in relatively large amounts and the size of the tablets is increased in proportion to such amounts. Under these circumstances, various attempts have been made to modify the surface characteristics of drugs at a molecular level utilizing a solid dispersion method. According to the solid dispersion method, particles are obtained by heating a mixture of a meltable additive and a drug or by using the solvent that can simultaneously dissolve two materials. In case of poorly water-soluble drugs, solubility is increased by the use of hydrophilic additives, such as polyethylene glycol and polyvinyl alcohol, to improve the wettability of the drugs, which leads to enhance the bioavailability. Meanwhile, in case of water-soluble drugs, the wettability is reduced by the use of hydrophobic additives, allowing the drugs to have sustained-release properties. Since the surface characteristics of drugs are modified at a molecular level by the solid dispersion method, additives can be used in minimum amounts to achieve maximum effects. Particularly the production procedure of preparations is simple, thus enabling practical production of the preparations in an efficient manner.

Production processes of preparations utilizing the solid dispersion method include melt-extrusion and melt-granulation processes. It is known that the melt-granulation process is suitable to produce sustained-release preparations. According to the melt-granulation process, physical force is applied to a mixture of a drug, at least one binder and an additive to attach the molten binder to the surface of the drug particles in order to produce granules. The mechanism of the melt-granulation process will now be specifically explained. After a drug, at least one binder and an additive are physically mixed, energy is applied to the mixture until the binder or the additive melts. Thereafter, the resulting mixture is cooled to form a solid lump and pulverized to obtain pellets having a desired size. The pellets are filled into a capsule, or mixed with another additive and compressed to produce a sustained-release preparation. U.S. Pat. No. 5,591,452 suggests a method for the production of a sustained-release preparation containing tramadol by the above melt-granulation process. On the other hand, the principle of the melt-extrusion process is similar as that of the melt-granulation process, except that melting, extrusion, cooling and pulverization are consecutively conducted in the melt-extrusion process. WO 93/15753 suggests a method for producing sustained-release pellets containing drugs by the above melt-extrusion process.

Solid dispersion methods using solvents are mainly utilized to solubilize drugs that are easily decomposed by heat or that have a relatively low solubility in water. Korean Patent No. 10-0396443 discloses a method for producing a sustained-release preparation of felodipine by the following procedures. A sparingly water-soluble felodipine and a solubilizer are dissolved in a co-solvent, spray-drying is conducted to obtain dispersion particles with improved water solubility, and the dispersion particles are mixed with a sustained-release base.

Current methods for producing sustained-release particles of drugs are largely classified into the following two groups: methods for imparting sustained-release properties to drug particles utilizing the solid dispersion method at a molecular level; and methods for producing sustained-release particles of drugs by coating drugs on inert beads and then coating the beads with a sustained-release coating material. U.S. Pat. Nos. 5,849,240, 5,891,471, 6,162,467, and 5,965,163 disclose methods for producing sustained-release preparations by forming sustained-release granules by the melt-granulation process and forming the granules into tablets or capsules. Further, U.S. Pat. Nos. 6,261,599, 6,290,990, and 6,335,033 disclose methods for producing sustained-release preparations by forming sustained-release pellets by the melt-extrusion process and forming the pellets into tablets. Meanwhile, U.S. Pat. Nos. 6,254,887 and 6,306,438 describes methods for producing sustained-release pellets by processes other than the melt-granulation and melt-extrusion processes. Specifically, sustained-release pellets are produced by the following procedures. First, a drug layer is coated on inert beads and then sustained-release coating layers are continuously formed thereon, or matrix pellets are produced using a wax-like binder and then a sustained-release coating layer is formed thereon. Secondly, a dispersion of a drug in a molten hydrophobic polymer is sprayed to produce pellets. Thirdly, matrix granules of a hydrophobic polymer and a drug are coated with a molten wax-like substance.

According to these methods, since the surface of a drug can be surrounded by a hydrophobic substance at a molecular level, effective release delay can be induced due to the presence of a small amount of a hydrophobic additive. Further, these methods are advantageous in terms of simple production procedure. Since most hydrophobic additives used in the melt-granulation and the melt-extrusion processes have characteristics similar to waxes, however, pellet particles obtained after melting and cooling tend to bind to the surface of other sites. Accordingly, flowability of the particles in a hopper is retarded upon compression into tablets, surface attachment of the particles to a punch and a die becomes severe, and resistance is increased when the tablets are removed from a tablet machine, causing serious problems in practical production of preparations. Although the surface attachment can be overcome by the addition of a lubricant to some extent, there is a limitation in reducing the surface attachment. Therefore, the amount of the hydrophobic additive used is limited. The lubricant is generally used in an amount of from about 0.1% to about 5%, based on the weight of the granules. When the amount of the lubricant is excessive, the release rate is delayed and capping and laminating take place upon compression into tablets. Meanwhile, when the amount of the lubricant is insufficient, chipping and picking arise.

U.S. Pat. Nos. 5,955,104, 5,968,551. 6,159,501 and 6,143,322, and PCT/EP1997/03934 teach methods for producing sustained-release pellets of a multiple unit dosage form by coating a drug layer on inert beads and then forming a coating layer composed of an alkylcellulose and an acrylic polymer thereon. Then, the pellets are filled into a capsule. It was observed that the effective blood level of an opioid analgesic was maintained for 24 hours. Particularly, U.S. Pat. No. 6,159,501 describes that the release rate can be controlled by mixing immediate-release uncoated pellets with sustained-release pellets, and by filling the mixture into a capsule. Further, U.S. Pat. Nos. 6,103,261 and 6,249,195 teach methods for producing sustained-release pellets by coating matrix pellets with an acrylic polymer and ethylcellulose in order to obtain the extension effect of duration of pain relief of about 24 hours, wherein the coating matrix pellets are composed of a gum, an alkylcellulose, an acrylic resin and a drug. Problems encountered with these methods are that two or more coating steps and particle blending step for subsequent release and content control of the drug are required, the overall volume of the particles is large in the case of preparations requiring a high drug content, and the sustained-release properties of the pellets are poor as compared to compressed tablets due to increased drug release areas of the pellets.

Sustained-release and controlled-release preparations of topiramate are taught in U.S. Patent Publication No. 2004/0115262 and U.S. Pat. No. 6,699,840. The preparation taught in U.S. Patent Publication No. 2004/0115262 is characterized by the use of an osmotic system and the inclusion of a surfactant to enhance solubility of the drug contained in a drug layer. The preparation comprises a drug layer containing the drug and the surfactant, and comprises an underlying expandable layer pushing the drug layer to release drug. Since topiramate has a large daily dose, the inclusion of the surfactant and the presence of the expandable layer render the overall size of the preparation large, substantially making it difficult to take the preparation. Solid-state topiramate present in the drug layer may clog drug-release pores, which results in inducing irregular drug release. In the case where the solid-state topiramate contained in the drug layer is transformed into a semi-solid or liquid state for smooth release of the topiramate, a considerable amount of the surfactant is needed and the volume of the preparation becomes larger. The preparation taught in U.S. Pat. No. 6,699,840 is characterized by the application of a topiramate salt for enhancing the solubility of the drug. Although increased drug solubility facilitates the control of drug release, the use of a larger amount of a sustained-release base material is required in order to induce comparable release delay effects, which disadvantageously increases the total weight of the preparation. Particularly, in the case of drugs, e.g., topiramate, having a daily maintenance dose of above 200 mg, the administration frequency is decreased due to enhanced solubility, but it is given more weight that increased weight of the drug preparations makes it difficult to swallow the preparations.

### DISCLOSURE OF THE INVENTION

Therefore, the present invention has been made in view of solving the above problems of the prior art. The present invention facilitates the control of release of drug by improving the wettability of the poorly water-soluble drug. Simultaneously, the present invention reduces the total weight of the drug preparation having a high daily dose by minimizing the addition of a release-sustaining material for imparting sustained-release properties. Ultimately therefore, the objects of the present invention are a decrease of administration frequency and enhancement of administration convenience.

The present invention relates to a sustained-release topiramate preparation and a method for producing the topiramate preparation.

In accordance with one aspect of the present invention, there is provided a sustained-release topiramate preparation produced using double granules obtained by granulating topiramate or a pharmaceutically acceptable salt thereof. The granulation is carried out using a solid dispersant by a solid dispersion method (first granulation), and further granulating the granules using a release-sustaining material by dry or wet granulation (second granulation).

It is preferred that the sustained-release preparation contains 0.5˜80% by weight of the drug, 1˜65% by weight of the solid dispersant, and 1˜55% by weight of the release-sustaining material, based on the total weight of the double granules.

As the solid dispersant, there can be used at least one material selected from the group consisting of polyvinylpyrrolidone, copovidone, polyethylene glycol, hydroxypropylmethylcellulose, Poloxamers, polyvinyl alcohol, cyclodextrin, hydroxyalkylcellulose phthalate, sodium cellulose acetate phthalate, cellulose acetyl phthalate, cellulose ether phthalate, anionic copolymers of methacrylic acid and methacrylic acid methyl or ethyl ester, hydroxypropylmethylcellulose phthalate, hydroxypropylmethylcellulose acetyl succinate, cellulose acetyl phthalate, and surfactants. It is preferred that the solid dispersant be hydrophilic.

Examples of the surfactant include, but are not particularly limited to, anionic surfactants, non-ionic surfactants, amphoteric surfactants, and mixtures thereof. Preferably, the surfactant can be selected from the group consisting of poly(oxyethylene) sorbitan fatty acid esters, poly(oxyethylene) stearate, poly(oxyethylene)alkyl ether, polyglycolated glyceride, poly(oxyethylene) castor oil, sorbitan fatty acid esters, Poloxamers, fatty acid salts, bile acid salts, alkyl sulfates, lecithin, mixed micelles of bile acid salts and lecithin, sugar ester vitamin E (polyethylene glycol 1000) succinate (TPGS), sodium lauryl sulfate, and mixtures thereof.

Preferred solid dispersants are polyvinylpyrrolidone, copovidone, hydroxypropylmethylcellulose, cellulose acetyl phthalate, polyvinyl alcohol, cyclodextrin, hydroxyalkylcellulose phthalate, Poloxaniers, sodium lauryl sulfate, and mixtures thereof. More preferred are polyvinylpyrrolidone, copovidone, hydroxypropylmethylcellulose, Poloxamers, sodium lauryl sulfate, and mixtures thereof.

Since the solid dispersant acts to uniformly surround the drug, sustained-release properties or solubility enhancement effects of the drug can be effectively achieved despite the use of a small amount of the solid dispersant. The solid dispersant used to produce the preparation of the present invention preferably has a melting point of 30˜150° C., and more preferably 50˜10° C.

As the release-sustaining material, there can be used at least one material selected from the group consisting of fatty acid alcohols, fatty acids, fatty acid esters, fatty acid glycerides, waxes, hydrogenated castor oil, hydrogenated vegetable oil, alkylcellulose, polyvinyl acetate, polyethylene oxide, hydroxypropylalkylcellulose, hydroxyalkylcellulose, sodium alginate, xanthan gum, locust bean gum, ammonio methacrylate copolymers, anionic copolymers of methacrylic acid and methacrylic acid methyl or ethyl ester, hydroxypropylmethylcellulose acetyl succinate, hydroxypropylmethylcellulose phthalate, and Carbopols. The release-sustaining material is attached to the surface of the primary granules obtained by a solid dispersion method to block the surface characteristics similar to those of waxes, and to induce the release delay of the drug.

Examples of suitable fatty acid alcohols include, but are not especially limited to, cetostearyl alcohol, stearyl alcohol, myristyl alcohol, and lauryl alcohol.

Examples of suitable fatty acids include, but are not especially limited to, oleic acid, myristic acid, linoleic acid, lauric acid, capric acid, caprylic acid, caproic acid, linolenic acid, and stearic acid.

Examples of suitable fatty acid esters include, but are not especially limited to, glyceryl monostearate, glycerol monooleate, acetylated monoglyceride, tristearin, tripalmitin, cetyl ester waxes, glyceryl palmitostearate, and glyceryl behenate.

Examples of suitable fatty acid glycerides include, but are not especially limited to, monoglyceride, diglyceride and triglyceride of linoleic acid and oleic acid, and monoglyceride, diglyceride and triglyceride of palmitic acid and stearic acid.

Examples of suitable waxes include, but are not especially limited to, beeswax, carnauba wax, glycowax, and castor wax.

Preferred release-sustaining materials are alkylcellulose, polyvinyl acetate, polyethylene oxide, hydroxypropylalkylcellulose, hydroxyalkylcellulose, sodium alginate, xanthan gum, locust bean gum, ammonio methacrylate copolymers, and mixtures thereof. More preferred are polyvinyl acetate, polyethylene oxide, hydroxypropylalkylcellulose, hydroxyalkylcellulose, sodium alginate, xanthan gum, locust bean gum, and mixtures thereof.

The sustained-release preparation of the present invention may further comprise at least one pharmaceutically acceptable additive selected from diluents, binders, swelling agents, lubricants, and other additives. The additive can be added in one or both steps of the first and second granulation steps. Particularly, the lubricant can be added during shaping or filling into the final unit preparation after the second granulation step.

Examples of suitable diluents include, but are not particularly limited to, lactose, dextrin, starch, microcrystalline cellulose, calcium hydrogen phosphate, anhydrous calcium hydrogen phosphate, calcium carbonate, saccharides, and the like.

Examples of suitable binders include, but are not particularly limited to, polyvinylpyrrolidone, copovidone, gelatin, starch, sucrose, methylcellulose, ethylcellulose, hydroxypropylcellulose, hydroxypropylalkylcellulose, and the like.

Examples of suitable swelling agents include, but are not particularly limited to, sodium alginate, crosslinked polyvinylpyrrolidone, carboxymethylcellulose (CMC), carboxymethylcellulose sodium (CMC-Na), carboxymethylcellulose calcium (CMC-Ca), starch, gelatin, Shellacs, liquorice powder, crystalline cellulose, calcium carbonate, sodium hydrogen carbonate, calcium phosphate, sodium lauryl sulfate, bentonite, sodium starch glycolate, tragacanth, methylcellulose, hydroxypropylmethylcellulose, and the like.

Examples of suitable lubricants include, but are not particularly limited to, stearic acid, stearic acid salts, talc, corn starch, carnauba wax, hard anhydrous silicic acid, magnesium silicate, synthetic aluminum silicate, hardened oil, white wax, titanium oxide, microcrystalline cellulose, Macrogols 4000 and 6000, myristic acid isopropyl, calcium hydrogen phosphate, talc, and the like.

The sustained-release preparation of the present invention may further comprise a coating layer containing a film-forming agent. The introduction of the coating layer facilitates the control over the release profile of the drug. This control over the release profile of the drug can be further performed by adjusting the thickness of the coating layer and the presence of a release-control material in the film-forming agent. As the release-control material, there may be used at least one material selected from the group consisting of saccharides, inorganic and organic salts, alkylcellulose, hydroxyalkylcellulose, hydroxypropylalkylcellulose, polyvinylpyrrolidone, polyvinyl alcohol, topiramate, and pharmaceutically acceptable topiramate salts. The coating layer included in the sustained-release preparation may contain topiramate and a pharmaceutically acceptable salt thereof in order to achieve an effective blood level as fast as possible after administration. The content of the drug in the coating layer is between 1% and 50%, and preferably between 1% and 20%, based on the total content of the drug in the preparation.

As the film-forming agent, there can be used at least one material selected from the group consisting of ethylcellulose, Shellacs, ammonio methacrylate copolymers, polyvinyl acetate, polyvinylpyrrolidone, polyvinyl alcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose, hydroxyalkylcellulose phthalate, sodium cellulose acetate phthalate, cellulose acetyl phthalate, cellulose ether phthalate, anionic copolymers of methacrylic acid and methacrylic acid methyl or ethyl ester, hydroxypropylmethylcellulose phthalate, hydroxypropylmethylcellulose acetyl succinate, cellulose acetyl phthalate, and Opadry (Colorcon Co.). Examples of the ammonio methacrylate copolymers include Eudragit RS™ and Eudragit RL™. Various effects, e.g., coloration, stability, dissolution control, prevention of initial excessive release of the drug and blocking of the drug taste can be achieved by coating with the film-forming agent.

The coating layer may further contain a plasticizer. In addition to the plasticizer, colorants, antioxidants, talc, titanium dioxide, flavoring agents, etc., can be used. The plasticizer may be at least one material selected from the group consisting of castor oil, fatty acids, substituted triglycerides and glycerides, triethyl citrate, and polyethylene glycol (molecular weight: 300˜50,000) and derivatives thereof.

Topiramate is poorly water-soluble and has a daily dose of 100 mg or more. Preparations of a drug having a high daily dose of 100 mg or more must have a size that is easy to take and continuously release the drug for a desired period of time by effective sustained release of the drugs. If topiramate is applied to a common sustained-release matrix, the amount of external fluids permeated into the matrix do not reach the dissolution and release levels of the drug and hence the drug is not sufficiently released while passing through the gastrointestinal tract. In addition, if the release-sustaining material is used in a smaller amount, the preparation is easily collapsed due to external factors, e.g., gastrointestinal motility, and thus satisfactory sustained-release functions are not exhibited.

The preparation of the present invention uses the solid dispersant and the release-sustaining material as additives for sustained release of topiramate. Further, the preparation of the present invention is produced by granulating topiramate using the solid dispersant (first granulation) and further granulating the granules using the release-sustaining material (second granulation), so that the molecular state and particle state of the drug are modified through the two steps. As a result, the amounts of the additives, i.e., the solid dispersant and the release-sustaining material, used can be markedly reduced.

When a sustained-release preparation of a poorly soluble drug is produced, the solubility of the drug contained in the preparation is drastically lowered, causing insufficient release of the drug within a desired period of time. According to the preparation of the present invention, the wettability of the drug is improved by the first granulation so that release of the drug from the preparation is smoothly induced, and at the same time, the release rate of the drug is controlled by the second granulation. Through those methods, the problems associated with low release rate and enlargement of the preparation can be solved.

In accordance with another aspect of the present invention, there is provided a method for producing the sustained-release preparations, comprising the steps of:

(1) mixing topiramate or a pharmaceutically acceptable salt thereof with a solid dispersant, and then subjecting to a solid dispersion method to obtain primary granules; and

(2) mixing the primary granules with a release-sustaining material, and then subjecting to dry or wet granulation to produce secondary granules.

The method of the present invention will now be explained in detail below. First, the drug is uniformly mixed with a solid dispersant by applying energy (heat) or adding a co-solvent thereto. The resulting mixture is cooled to below a temperature sufficient to melt or soften the solid dispersant, or the solvent is evaporated using a spray dryer, a fluid bed spray coater or a vacuum evaporator to obtain primary solid granules. If necessary, pharmaceutically acceptable additives, such as diluents, binders and swelling agents, can be added during the first granulation. After the primary granules are pulverized to a constant size and sieved, a release-sustaining material is added to the sieved granules. Thereafter, the mixture is subjected to second granulation to produce the final sustained-release preparation. Pharmaceutically acceptable additives, such as diluents, binders and swelling agents, may be added during the second granulation. The sustained-release preparation is filled into a capsule or compressed into a tablet.

The method of the present invention may further comprise the step of coating the secondary granules or the tablet obtained by compressing the granules, with a coating solution containing a film-forming agent. As a solvent of the coating solution for forming a coating layer, water or an organic solvent can be used. Examples of preferred organic solvents include methanol, ethanol, isopropanol, acetone, chloroform, dichloromethane, and mixtures thereof.

### BRIEF EXPLANATION OF DRAWINGS

The above and other objects, features and other advantages of the present invention will be more clearly understood from the following detailed description taken in conjunction with the accompanying drawing, in which:

FIG. 1 is a graph showing the results of the dissolution test on sustained-release preparations produced in Examples 1 (□), 3 (▪), 5 (▴), and 6 (•) and Comparative Example 1 (♦).

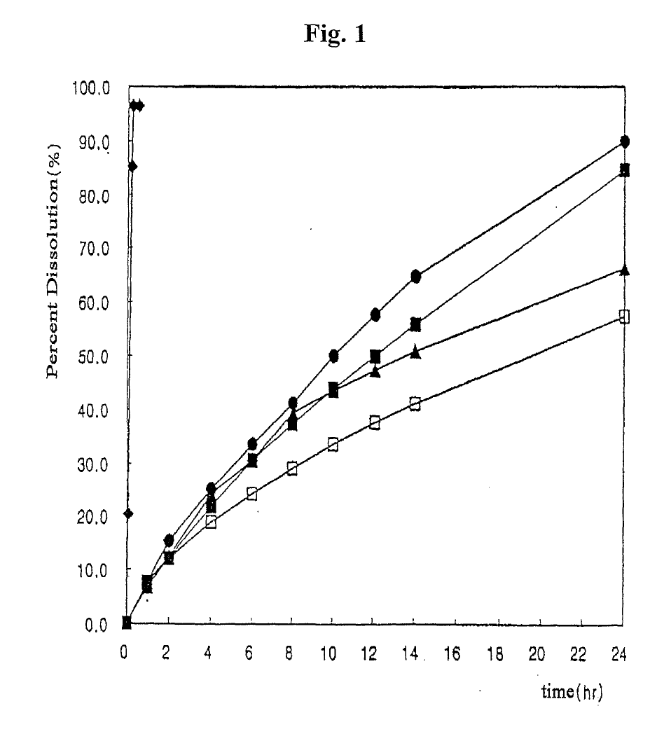

### MODE FOR CARRYING OUT THE INVENTION

The present invention will now be described in more detail with reference to the following examples and experimental examples. However, these examples are not to be construed as limiting the scope of the invention.

### Examples 1 to 3

### Production of Matrix Tablets Containing Topiramate

Glyceryl behenate and topiramate were mixed under heating to 70° C. until the glyceryl behenate was melted or softened. The mixture was cooled to room temperature to form a solid lump. Thereafter, the solid lump was pulverized and passed through a 20-mesh sieve. The particles passed through the sieve were mixed with the additives shown in Table 1, and each of the mixtures was subjected to wet granulation (second granulation). The obtained granules were dried, and then magnesium stearate was added thereto. The mixtures were compressed into respective tablets. The matrix tablets had the respective compositions shown in Table 1.

### Comparative Example 1

A topiramate preparation currently sold under the trade name TOPAMAX® (100 mg, Janssen Korea, Ltd.) was used as comparative Example 1.

### Comparative Example 2

Glyceryl behenate and topiramate were mixed under heating to 70° C. until the glyceryl behenate was melted or softened. The mixture was cooled to room temperature to form a solid lump. Thereafter, the solid lump was pulverized and passed through a 20-mesh sieve. The particles passed through the sieve were mixed with the additives shown in Table 1, and then compressed to an appropriate size to produce a tablet.

### Comparative Example 3

A mixture of glyceryl behenate and topiramate was mixed with the additives shown in Table 1 without formation of a solid dispersion, and the resulting mixture was subjected to wet granulation. The tablet was produced by the same procedure as Example 1. The matrix tablet had the composition shown in Table 1.

TABLE 1
Compositions of matrix tablets

| | | | | Comparative | Comparative |
|---|---|---|---|---|---|
| Ingredients (mg) | Example 1 | Example 2 | Example 3 | Example 2 | Example 3 |
| Topiramate | 200 | 200 | 200 | 200 | 200 |
| Glyceryl behenate | 105 | 70 | 35 | 105 | 35 |
| Polyvinyl acetate | 24.8 | 42 | 42 | — | 42 |
| Polyvinylpyrrolidone | 16.7 | 21 | 21 | — | 21 |
| Microcrystalline | — | 13.5 | 48.5 | 41.5 | 48.5 |
| cellulose | | | | | |
| Magnesium stearate | 3.5 | 3.5 | 3.5 | 3.5 | 3.5 |
| Water* | q.s. | q.s. | q.s. | q.s. | q.s. |
| Total | 350 | 350 | 350 | 350 | 350 |

*Removed during production

### Experimental Example 1

### Surface Attachment Test

Solid dispersions were prepared from the tablets produced in Example 1 and Comparative Example 2 using solid dispersants having the same amount in accordance with the same procedure. Since the solid dispersion prepared from the tablet of Example 1 could block surface attachment of the primary granules by the second granulation, no attachment to the surface of a tablet punch or a die was observed upon compression. The granules produced in Comparative Example 2 showed severe surface attachment despite the addition of a lubricant, and as a result, the production of a tablet was impossible.

### Experimental Example 2

### Dissolution Test

Release profiles of the matrix tablets produced in Examples 1 to 3 and Comparative Example 3 and release profiles of the preparation of Comparative Example 1 were observed using a USP dissolution tester. The percent dissolution of the drug from the tablets was measured was measured as function of time under the following condition: pH 6.8, phosphate buffer, Paddle method, 50 rpm/900 ml. The results as shown in Table 2.

TABLE 2
Percent dissolution (%) with the passage of time

| Time | Exam- | Exam- | Exam- | Comparative | Time | Comparative |
|---|---|---|---|---|---|---|
| (hr) | ple 1 | ple 2 | ple 3 | Example 3 | (min.) | Example 1 |
| 0 | 0.0 | 0.0 | 0.0 | 0.0 | 0.0 | 0.0 |
| 1 | 7.6 | 11.6 | 6.7 | 38.47 | 5 | 20.3 |
| 2 | 12.2 | 16.2 | 12.3 | 54.57 | 10 | 85.3 |
| 4 | 18.9 | 22.9 | 21.8 | 74.09 | 15 | 96.5 |
| 6 | 24.3 | 28.3 | 30.6 | 84.14 | 30 | 96.4 |
| 8 | 29.1 | 33.6 | 37.4 | 88.02 | — | — |
| 10 | 33.4 | 37.4 | 43.7 | — | — | — |
| 12 | 37.5 | 41.5 | 50.1 | — | — | — |
| 14 | 41.1 | 45.1 | 55.9 | — | — | — |
| 24 | 57.5 | 61.5 | 84.5 | — | — | — |

Results of the dissolution test on the tablets of Comparative Example 1 and Examples 1 to 3 indicate that the drug was slowly released for 24 hours or longer by the double granulation. From the results of the dissolution test on the tablets of Comparative Example 3 and Example 3, it could be confirmed that the surface characteristics of the drug were changed by the solid dispersion method, leading to effective release delay. Further, the results represents that since comparable release delay effects can achieved by the use of a small amount of the solid dispersant or the release-sustaining material, release delay of the drug can be induced without any increase in the total weight of the preparations. On the other hand, the solid dispersion could block surface attachment by the second granulation, and thus the production of the tablets was easy. As can be seen from the results of the dissolution test on the tablets produced in Examples 1 to 3, the release rates of the drug could be controlled by controlling the amount of the solid dispersant.

The release levels of the drug from the tablets produced in Examples 1 to 3 were reduced to below 90% after 24 hours, which indicates effective sustained release. However, the drug was not sufficiently released from the inside of the matrices within the period due to low solubility of topiramate.

### Examples 4 to 7

### Production of Matrix Tablets Containing Topiramate

Glyceryl behenate and topiramate were mixed under heating to 70° C. until the glyceryl behenate was melted or softened. The mixture was cooled to room temperature to form a solid lump. Thereafter, the solid lump was pulverized and passed through a 20-mesh sieve. The particles passed through the sieve were mixed with the additives shown in Table 3, and each of the mixtures was subjected to dry granulation. Magnesium stearate was added to the granules, mixed, and compressed into appropriate forms to produce tablets. The matrix tablets had the respective compositions shown in Table 3.

TABLE 3
Compositions of matrix tablets

| Ingredients (mg) | Example 4 | Example 5 | Example 6 | Example 7 |
|---|---|---|---|---|
| Topiramate | 200 | 200 | 200 | 200 |
| Glyceryl behenate | 35 | 35 | 35 | 35 |
| Polyvinyl acetate | 56 | 56 | 56 | 112 |
| Polyvinylpyrrolidone | 24.5 | 35 | 49 | 28 |
| Microcrystalline | 31 | 20.5 | 6.5 | 1.2 |
| cellulose | | | | |
| Magnesium stearate | 3.5 | 3.5 | 3.5 | 3.8 |
| Total | 350 | 350 | 350 | 380 |

### Experimental Example 3

### Dissolution Test

The percent dissolution of the drug from the matrix tablets produced in Examples 4 to 7 were measured as a function of time by the same procedure as in Experimental Example 2. The results are shown in Table 4.

TABLE 4
Percent dissolution (%) with the passage of time

| Time (hr) | Example 4 | Example 5 | Example 6 | Example 7 |
|---|---|---|---|---|
| 0 | 0.0 | 0.0 | 0.0 | 0.0 |
| 1 | 6.9 | 7.7 | 7.8 | 8.9 |
| 2 | 11.4 | 12.4 | 15.3 | 13.4 |
| 4 | 17.9 | 24.1 | 25.2 | 19.9 |
| 6 | 22.9 | 30.5 | 33.4 | 24.8 |
| 8 | 27.3 | 39.2 | 41.2 | 28.9 |
| 10 | 31.2 | 43.5 | 50.0 | 32.5 |
| 12 | 34.7 | 47.3 | 57.6 | 35.7 |
| 14 | 38.0 | 51.0 | 64.9 | 38.4 |
| 24 | 54.3 | 66.2 | 90.0 | 48.2 |

As apparent from the results of the dissolution test on the tablets produced in Examples 3, 4 and 7, the release rates of topiramate can be controlled by the amount of the release-sustaining material upon the second granulation. Results of the dissolution test on the tablets produced in Examples 4 to 6 represent that since the hydrophilic binder acts as a pore for drug release in the matrices, the release of the drug increases with increasing content of the hydrophilic binder.

### Example 8

### Production of Coated Matrix Tablet Containing Topiramate

Copovidone and topiramate were uniformly mixed in anhydrous ethanol as a co-solvent, and then the solvent was evaporated to form a solid dispersion. The solid dispersion was passed through a 20-mesh sieve. The particles passed through the sieve were mixed with the additives shown in Table 5, and the mixture was subjected to dry granulation (second granulation). Magnesium stearate was added to the granules, mixed, and compressed into appropriate forms to produce a tablet. The matrix tablet was coated with a coating solution having the composition indicated in Table 5 by spray coating using a fan coater, and dried to produce a coated matrix tablet.

TABLE 5
Composition of matrix tablet and coating solution

| Composition | Ingredients (mg) | Example 8 |
|---|---|---|
| Matrix | Topiramate | 200 |
| | Copovidone | 76 |
| | Polyvinyl acetate | 60.8 |
| | Polyvinylpyrrolidone | 15.2 |
| | Lactose | 24.2 |
| | Magnesium stearate | 3.8 |
| | Anhydrous ethanol* | q.s. |
| Coating | Opadry (AMB 80W | 15.2 |
| solution | 42096 Yellow) | |
| | Purified water* | 70 |
| Total | | 395.2 |

*Removed during production

### Experimental Example 4

### Dissolution Test

The percent dissolution of the drug from the matrix tablet produced in Example 8 were measured as a function of time by the same procedure as in Experimental Example 2. The results are shown in Table 6.

TABLE 6
Percent dissolution (%) with the passage of time

| Time (hr) | Example 8 |
|---|---|
| 0 | 0.0 |
| 1 | 16.1 |
| 2 | 25.3 |
| 4 | 38.8 |
| 6 | 48.3 |
| 8 | 56.0 |
| 10 | 62.4 |
| 12 | 68.1 |
| 14 | 73.3 |
| 18 | 81.0 |
| 24 | 90.0 |

From the results of the dissolution test on the tablet produced in Example 8, it could be confirmed that topiramate was continuously released for 24 hours or longer.

### Examples 9 to 13

### Production of Matrix Tablets Containing Topiramate

Topiramate, lactose and polyvinylpyrrolidone were uniformly mixed in anhydrous ethanol, and then the solvent was evaporated to form a solid dispersion. In Examples 11 to 13, sodium lauryl sulfate or carboxymethylcellulose sodium (CMC-Na) was further added. The dried primary granules were passed through a 20-mesh sieve. The particles passed through the sieve were mixed with the additives shown in Table 7, and each of the mixtures was subjected to dry granulation (second granulation). Magnesium stearate was added to the granules, mixed, and compressed into appropriate forms to produce respective tablets.

TABLE 7
Compositions of matrix tablets

| | Exam- | Example | Exam- | Exam- | Exam- |
|---|---|---|---|---|---|
| Ingredients (mg) | ple 9 | 10 | ple 11 | ple 12 | ple 13 |
| Topiramate | 200 | 200 | 200 | 200 | 200 |
| Lactose | 24.2 | 24.2 | 39.4 | 48.4 | 50 |
| Lauryl sodium | — | — | 11.4 | 22.8 | 12 |
| sulfate | | | | | |
| Polyvinylpyrrolidone | 15.2 | 11.4 | 25.2 | 30.4 | 26.8 |
| CMC-Na | — | — | — | — | — |
| Polyvinyl acetate | 60.8 | 45.6 | 91.2 | 91.2 | 91.2 |
| Copovidone | 76 | 95 | 9 | 3.8 | 12 |
| Magnesium stearate | 3.8 | 3.8 | 3.8 | 3.4 | 4 |
| Anhydrous ethanol* | 25 | 25 | 25 | 35 | 26 |
| Total (mg) | 380 | 380 | 380 | 400 | 400 |

*Removed during production

### Experimental Example 5

### Dissolution Test

The percent dissolution of the drug from the matrix tablets produced in Examples 9 to 13 were measured as a function of time by the same procedure as in Experimental Example 2. The results are shown in Table 8.

TABLE 8
Percent dissolution (%) with the passage of time

| Time | | Time | Example | Time | Example | Time | Example | Time | Example |
|---|---|---|---|---|---|---|---|---|---|
| (hr) | Example 9 | (hr) | 10 | (hr) | 11 | (hr) | 12 | (hr) | 13 |
| 0 | 0.00 | 0 | 0.00 | 0 | 0.00 | 0 | 0.00 | 0 | 0.00 |
| 1 | 18.20 | 1 | 20.32 | 1 | 14.62 | 1 | 14.02 | 1 | 17.96 |
| 2 | 27.49 | 2 | 32.45 | 2 | 22.07 | 2 | 23.74 | 2 | 32.70 |
| 4 | 40.90 | 4 | 48.79 | 3 | 27.79 | 3 | 34.31 | 3 | 45.16 |
| 6 | 50.43 | 6 | 63.40 | 4 | 32.97 | 4 | 44.27 | 4 | 54.78 |
| 8 | 58.21 | 8 | 75.55 | 7 | 48.93 | 7 | 66.65 | 7 | 74.99 |
| 10 | 64.63 | 10 | 84.74 | 10 | 63.51 | 10 | 79.39 | 10 | 87.20 |
| 12 | 70.34 | 12 | 90.79 | 14 | 78.97 | 14 | 86.98 | 14 | 92.92 |
| 14 | 75.47 | | | 16 | 84.77 | 16 | 88.07 | 16 | 93.19 |
| 18 | 83.19 | | | | | | | 24 | 97.19 |
| 24 | 92.28 | | | | | | | | |

As can be seen from the results of the dissolution test on the tablets produced in Examples 9 and 10, the release rates of the drug could be controlled by controlling the content of the release-sustaining material contained in the second granulation. In addition, the results of the dissolution test on the tablets produced in Examples 11 and 12 indicate that the presence of the surfactant increases the dissolution rate of topiramate contained in the sustained-release matrices, leading to an increase in the release rate of the drug. From comparison between the results of the dissolution test on the tablets produced in Examples 11 and 13, it could be confirmed that the addition of the swelling agent to the sustained-release matrices can increase the diffusion release rates of the drug through the drug matrices.

### Examples 14 and 15

### Production of Coated Matrix Tablets Containing Topiramate

The matrix tablet produced in Example 13 was coated with coating solutions having the respective compositions indicated in Table 9 by spray coating using a fan coater, and dried to produce coated matrix tablets.

TABLE 9
Composition of coating solutions

| Ingredients (mg) | Example 14 | Example 15 |
|---|---|---|
| Hydroxypropylmethylcellulose 2910 | 12 | 16 |
| Ethylcellulose 7 cp | 8 | 4 |
| Triethyl citrate | 2 | 2 |
| Ethanol* | 275.73 | 275.73 |
| Purified water* | 68.93 | 68.93 |

*Removed during production

### Experimental Example 6

### Dissolution Test

The percent dissolution of the drug from the matrix tablets produced in Examples 14 and 15 were measured as a function of time by the same procedure as in Experimental Example 2. The results are shown in Table 10.

TABLE 10
Percent dissolution (%) with the passage of time

| Time (hr) | Example 14 | Example 15 |
|---|---|---|
| 0 | 0.00 | 0.00 |
| 1 | 7.37 | 12.78 |
| 2 | 17.43 | 25.63 |
| 3 | 25.87 | 35.96 |
| 4 | 34.14 | 45.20 |
| 7 | 56.64 | 66.18 |
| 10 | 71.04 | 79.99 |
| 14 | 82.45 | 89.89 |
| 16 | 85.91 | 92.69 |

The results of the dissolution test on the tablets of Examples 14 and 15 confirm that the introduction of the coating layers can delay the initial release rate of the drug.

### Examples 16 and 17

### Production of Matrix Tablets Containing Topiramate

In Example 16, topiramate, lactose, polyvinylpyrrolidone, sodium lauryl sulfate and crosslinked polyvinylpyrrolidone were uniformly mixed in anhydrous ethanol, and then the solvent was evaporated to form a primary granule. In Example 17, topiramate, copovidone and microcrystalline cellulose were mixed in anhydrous ethanol to form a primary granule. The dried primary granules were passed through a 20-mesh sieve. The particles passed through the sieve were mixed with the additives shown in Table 11 and each of the mixtures was subjected to dry granulation (second granulation). Magnesium stearate was added to the granules, mixed, and compressed into appropriate forms to produce respective tablets.

TABLE 11

| Ingredients (mg) | Example 16 | Example 17 |
|---|---|---|
| Topiramate | 200 | 200 |
| Lactose | 56.4 | — |
| Lauryl sodium sulfate | 12 | — |
| Polyvinylpyrrolidone | 24.52 | — |
| Crosslinked polyvinylpyrrolidone | 9 | — |
| Polyvinyl acetate | 82.08 | — |
| Copovidone | 12 | 16 |
| Hydroxypropylmethylcellulose | — | 51.90 |
| Xanthan gum | — | 18.35 |
| Microcrystalline cellulose | — | 104.9 |
| Calcium hydrogen phosphate | — | 54.9 |
| Magnesium stearate | 4 | 4 |
| Anhydrous ethanol* | 26 | 30 |
| Total (mg) | 400 | 400 |

*Removed during production

### Experimental Example 7

### Dissolution Test

Release profiles of the matrix tablets produced in Examples 16 and 17 were observed using a USP dissolution tester. The percent dissolution of the drug from the tablets were measured as a function of time under the following conditions: pH 6.8, phosphate buffer, Paddle method, 75 rpm/900 ml. The results are shown in Table 12.

TABLE 12
Percent dissolution (%) with the passage of time

| Time (hr) | Example 16 | Example 17 |
|---|---|---|
| 0 | 0.00 | 0.00 |
| 1 | 22.64 | 22.64 |
| 2 | 35.34 | 35.34 |
| 3 | 45.08 | 45.08 |
| 4 | 53.29 | 53.29 |
| 7 | 71.28 | 71.28 |
| 10 | 84.81 | 84.81 |
| 14 | 95.96 | 95.96 |
| 16 | 98.11 | 98.11 |

As can be seen from the data shown in Table 12, the release rates of topiramate from the tablets produced in Examples 16 and 17 are expressed as a first-order function and a zero-order function of the release time, respectively. The relationship between the percent dissolution and dissolution time in Examples 16 and 17 can be represented by the following equations, respectively. Correlation coefficients (R—square values) in each equation were determined to be 98.4% and 95.5%.
Percent dissolution of topiramate (%) in Example 16=8.054+12.53×(dissolution time)(hr)−0.4379×(dissolution time)²(hr²) (1)
Percent dissolution of topiramate (%) in Example 17=3.859+5.375×(dissolution time)(hr) (2)

### INDUSTRIAL APPLICABILITY

The sustained-release topiramate preparation of the present invention can continuously release topiramate for 12 hours or longer to maintain the effective blood level of the drug for a long period of time. In addition, although the sustained-release preparation of the present invention contains topiramate having a high daily dose, it has a size that is easy to take, can provide convenience to patients. Furthermore, since the production procedure of the preparation is simple and surface attachment of the granules is markedly reduced, the preparation can be easily produced.

1. A sustained-release topiramate preparation produced using double granules obtained by a process comprising the steps of,
granulating topiramate or a pharmaceutically acceptable salt thereof using a solid dispersant by a solid dispersion method (first granulation); and further
granulating the resultant granules using a release-sustaining material by a dry or a wet granulation process (second granulation).

2. The preparation according to claim 1, wherein the preparation contains 0.5˜80% by weight of topiramate or a pharmaceutically acceptable salt thereof, 1˜65% by weight of the solid dispersant, and 1˜55% by weight of the release-sustaining material, based on the total weight of the double granules.

3. The preparation according to claim 1, wherein the solid dispersant is at least one material selected from the group consisting of polyvinylpyrrolidone, copovidone, polyethylene glycol, hydroxypropylmethylcellulose, Poloxamers, polyvinyl alcohol, cyclodextrin, hydroxyalkylcellulose phthalate, sodium cellulose acetate phthalate, cellulose acetyl phthalate, cellulose ether phthalate, anionic copolymers of methacrylic acid and methacrylic acid methyl or ethyl ester, hydroxypropylmethylcellulose phthalate, hydroxypropylmethylcellulose acetyl succinate, cellulose acetyl phthalate, and surfactants.

4. The preparation according to claim 3, wherein the surfactant is at least one material selected from the group consisting of poly(oxyethylene) sorbitan fatty acid esters, poly(oxyethylene) stearate, poly(oxyethylene)alkyl ether, polyglycolated glyceride, poly(oxyethylene) castor oil, sorbitan fatty acid esters, Poloxamers, fatty acid salts, bile acid salts, alkyl sulfates, lecithin, mixed micelles of bile acid salts and lecithin, sugar ester vitamin E (polyethylene glycol 1000) succinate (TPGS), and sodium lauryl sulfate.

5. The preparation according to claim 1, wherein the release-sustaining material is at least one material selected from the group consisting of fatty acid alcohols, fatty acids, fatty acid esters, fatty acid glycerides, waxes, hydrogenated castor oil, hydrogenated vegetable oil, alkylcellulose, polyvinyl acetate, polyethylene oxide, hydroxypropylalkylcellulose, hydroxyalkylcellulose, sodium alginate, xanthan gum, locust bean gum, ammonio methacrylate copolymers, anionic copolymers of methacrylic acid and methacrylic acid methyl or ethyl ester, hydroxypropylmethylcellulose acetyl succinate, hydroxypropylmethylcellulose phthalate, and Carbopols.

6. The preparation according to claim 1, further comprising a pharmaceutically acceptable additive selected from diluents, binders, swelling agents, lubricants, and other additives.

7. The preparation according to claim 6, wherein the swelling agent is at least one material selected from the group consisting of sodium alginate, crosslinked polyvinylpyrrolidone, carboxymethylcellulose (CMC), carboxymethylcellulose sodium (CMC-Na), carboxymethylcellulose calcium (CMC-Ca), starch, gelatin, Shellacs, liquorice powder, crystalline cellulose, calcium carbonate, sodium hydrogen carbonate, calcium phosphate, sodium lauryl sulfate, bentonite, sodium starch glycolate, tragacanth, methylcellulose, and hydroxypropylmethylcellulose.

8. The preparation according to claim 1, further comprising a coating layer containing a film-forming agent.

9. The preparation according to claim 8, wherein the coating layer further contains a release-control material, and the release-control material includes at least one material selected from the group consisting of saccharides, inorganic salts, organic salts, alkylcellulose, hydroxyalkylcellulose, hydroxypropylalkylcellulose, polyvinylpyrrolidone, polyvinyl alcohol, topiramate, and pharmaceutically acceptable topiramate salts.

10. The preparation according to claim 8, wherein the coating layer contains the drug in an amount of 1˜50%, based on the total amount of the drug in the preparation.

11. The preparation according to claim 8, wherein the film-forming agent is at least one material selected from the group consisting of ethylcellulose, Shellacs, ammonio methacrylate copolymers, polyvinyl acetate, polyvinylpyrrolidone, polyvinyl alcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose, hydroxyalkylcellulose phthalate, sodium cellulose acetate phthalate, cellulose acetyl phthalate, cellulose ether phthalate, anionic copolymers of methacrylic acid and methacrylic acid methyl or ethyl ester, hydroxypropylmethylcellulose phthalate, hydroxypropylmethylcellulose acetyl succinate, cellulose acetyl phthalate, and Opadry (Colorcon Co.).

12. A method for producing the sustained-release preparations according to claim 1, comprising the steps of:
(1) mixing topiramate or a pharmaceutically acceptable salt thereof in the amount of effective dose with a solid dispersant, and then obtaining primary granules by a solid dispersion method; and
(2) mixing the primary granules with a release-sustaining material, and then producing secondary granules by a dry or a wet granulation process.

13. The method according to claim 12, further comprising the step of coating the secondary granules or a tablet obtained by compressing the same granules, with a coating solution containing a film-forming agent.
